(19) United States
(12) Patent Application Publication
(10) Pub. No.: US 2020/0090807 A1
(43) Pub. Date: Mar. 19, 2020

(54) WIRELESS HEAD MOUNTED TELEMETRY DISPLAY AND COMMUNICATION SYSTEM

(71) Applicant: The Unites States of America Represented by the Secretary of Navy, Silver Spring, MD (US)

(72) Inventors: Jacob H. Cole, Suffolk, VA (US); Scott B. Hughey, Norfolk, VA (US)

(21) Appl. No.: 16/453,969

(22) Filed: Jun. 26, 2019

Related U.S. Application Data
(60) Provisional application No. 62/689,974, filed on Jun. 26, 2018.

(51) Int. Cl. G16H 40/67; A61N 1/372; G02B 27/01

(52) U.S. Cl. CPC G16H 40/67; A61N 1/37247; G02B 2027/014; A61N 1/37282; G02B 27/017

(57) ABSTRACT

The present invention discloses a patient monitoring and communication system, which allows remote display of real-time patient clinical and monitoring parameters, and supports selective hand-free communications between members of a multisite medical team.

### CROSS-REFRENCE TO RELATED APPLICATIONS

This application claims priority to U.S. Provisional Application 62/689,974, filed on Jun. 26, 2018.

### BACKGROUND

The present invention relates to a medical telemetry system, which collects patients' clinical information, remotely transmits the collected clinical information to a hub or one or more computer terminals, and displays a patient's medical information on selectively operable heads-up systems.

As the fields of anesthesiology, trauma resuscitation, and critical care advance, physicians are called upon to monitor, and synthesize an increasing number of clinical parameters. The traditional anesthesia machine provides controls over the flow and mixtures of oxygen, and anesthetic to a patient. The anesthesia machine also have gauges and indicators for the monitoring of the patient's clinical parameters, such as flow rates, supply pressures, respiratory volumes, patient temperature, blood pressure, pulse rate, oxygen and carbon dioxide concentrations as well as electrocardiographic data. Other physiology monitors are also routinely used in an operation to provide additional information informing the physician of what is happening to a patient. For example, a lung-water computer monitors extra vascular lung water. An electroencephalogram (EEG) computer displays the patient's EEGs in real-time during surgical procedures.

While adding great values to patient monitoring, incorporating these clinical parameters into their practice significantly compounded the complexity of the anesthetist's workload. To further complicating the situation, these monitors are often distributed about the operating room. Therefore, make it difficult for the anesthetist to scan the readouts and indicators of all monitors visually. Throughout the operation, the anesthetist must occasionally make adjustments to the anesthetic, oxygen flow control, intravenous lines, and monitoring equipment, which requires movement away from the head of the operating table, and subsequent reorientation to the monitor readouts. Under the current standard of care, providers in these clinical situations are reliant upon static wall or gimbal mounted displays to display pertinent physiologic parameters via visual graphic and auditory cues. Oftentimes in critical periods of care, an anesthesiologist's attention is drawn from these displays to attend to a vital task such as line placement, airway management, peripheral nerve blockade, medication administration, and fluid/blood product administration, etc. Even in periods of relative stability, a provider's attention is often drawn from these monitor displays by the necessity of medical documentation. In these instances, the provider is required to intermittently redirect their attention from the task at hand to focus on the physiologic monitors. The static nature of these standard displays often requires whole body movement, and not just a simple aversion of the eyes. Distraction like this introduces significant risks for procedural, medication, or documentation errors.

In health care field, the current trend is to transition many skilled functions away from physicians to physician extenders, such as technicians and nurses under the supervision of physicians. In the field of anesthesiology, nurse anesthetists are employed for many surgical procedures. However, it is often impractical for an anesthesiologist to adequately supervise multiple nurse anesthetists or anesthesiologists in training in simultaneous operations due to the physical layout of surgical suits of a hospital. While existing display system allows for mirroring of intraoperative monitors to an outside monitor, the locations of these outside monitors are static. A supervising anesthesiologist is required to remain at a fixed location to monitor the patients under the care of his or her physician extenders/residents. However, physician anesthesiologists are often engaged in patient care activities themselves in either the pre-operative area or the post-anesthesia care unit (PACU), which take them away from direct monitoring of intraoperative monitors of the patients under the care of their physician extenders. As a result, the physician extenders often only notify the physician anesthesiologist of critical intra-operative events. It is often difficult for the physician anesthesiologist to immediately attend to emergency situations in another operation suit while performing direct patient care, such as obtaining pre-operative intravenous access, performing pre-operative peripheral nerve blockade, or to performing post-operative patient evaluations.

Further, in critical moments of patient care, the noise level in the environment can also escalate to such a point that it is difficult to perceive the auditory cues that are being provided by conventional monitors. Currently, communications between different members of a critical care team is still depended on direct in person conversations. For example, in the perioperative environment, this can lead to delays as technicians, nurses, and anesthesia providers each prepare within their respective roles for the next case. Miscommunications can also cause a medication or a piece of equipment being inadvertently omitted or utilized. In the intensive care environment, nursing support staff have to leave a patient's bedside in order to inform an intensivist or representative of a clinical concern, a potential risk in the delivery of care to a critically ill patient. Intensivists often rely on traditional desk phones to communicate with the nursing staff Failing this, they must leave their workstation to discuss a case with the nurse assigned to a patient, interrupting their workflow, and adding a distractor to the provision of their care. In the trauma setting, a team leader must rely on ancillary support to order critical blood products, imaging, or lab studies. If they were to attend to these tasks themselves, it would involve the use of a computer workstation or phone, which would intermittently remove themselves from the patient care environment, and potentially impeding the efficient delivery of care in a critical setting.

Austere environments present their own host of challenges in the delivery of healthcare. Specifically in military environments, care is delivered in sub-optimal circumstances where a provider is stretched beyond their typical clinical capacity. It is the standard practice for medical evacuation teams to work to stabilize a critically ill casualty in small, noisy and constantly moving environment, with randomly placed portable monitors, which require a heightened level of vigilance. It is not uncommon to not observe a critical casualty's vital signs for minutes at a time. In an in-theater operating room or trauma bay, a physician anesthesiologist may be called on to directly care for more than one patient at once. While this happens rarely, it theoretically requires the obvious splitting of attention between two critical patients who may be in different rooms undergoing surgery at the same time. The clinical challenges to the practice of safe and effective care are obvious in these situations, and can stand to be improved through an improvement in the physiologic monitoring process.

Therefore, there is an unmet need for a patient monitoring and communication system, which allows remote monitoring of real-time patient clinical parameters and machine readouts, and supports selective hand-free communications between members of a multisite medical team.

### SUMMARY OF THE INVENTION

Accordingly, it is an objective of this invention to provide an improved patient monitoring and communication system for the care of one or more anesthetized patients. The patient monitor system of the present invention comprises a head mounted display and communication unit, which could display in real-time anesthesiology machine parameters and vital signs, as well as medical records of selective patients to a care provider, and to allow hand-free communications between members of different patient care teams at different locations.

The primary advantage of this system when compared to currently available products comes from decreasing distractions during critical moments of patient care. By utilizing a head mounted display, and communication device (HMD) to display physiologic parameters that were previously relegated to a statically mounted monitor, care providers can maintain focus on the task at hand without interruption. By allowing clinical parameters and medical histories to always be within a provider's direct field of vision, changes in patient's vital signs can be noticed quicker, and care can be given in a more efficient and rapid fashion.

Another advantage of the present invention is that functions of the inventive monitor and communication system can be voice controlled. By allowing for voice activated control and calling, perioperative teams will have the benefit of constant, real time indirect communication regarding upcoming cases. Questions can be answered, and uncertainties can be voiced, and resolved before errors occur. Physicians can be consulted by nursing staff without ever leaving the patient's bedside. Physicians can communicate directly with ancillary support staff such as the radiology department, the blood bank, or other consulting physicians while still maintaining hands on care of a patient. A surgeon will be able to keep his eyes on the surgical field while still constantly assessing a patient's vital signs, just as a nurse will be able to focus on their documentation while constantly monitoring their patient's hemodynamics.

Efficiency in the acute care setting is further improved by the present system through the integration of VoIP functionality. This system maintains a dedicated VoIP system throughout all functional areas within range of the wireless network, which can be integrated into a hospital's hardwired phone line system to allow calling throughout the facility. Communication between members of a care team is facilitated by a microphone, and the use of a bone conduction headset integrated into the HMD. Bone conduction headsets allow for maintenance of native hearing, and situational awareness while still providing easily perceivable audio cues provided by conventional monitors to the provider wearing the HMD despite the ambient noise level. The present invention provides physicians with the ability to provide direct supervision over their physician extenders remotely. For instance, in the perioperative environment, an anesthesia provider who is directly caring for a patient in an operating room will be able to rapidly consult the physician anesthesiologist who is overseeing care in the surgical center regarding any intraoperative concerns. By simply asking the device to contact their supervisor, this system allows the physician outside the room to immediately bring up the physiologic parameters of the patient in question as well as communicate directly with the in-room provider. Currently, the physician extender would need to contact the overseeing physician by either a hospital-issued or personal communication device (often a cell phone), and then the provider would need to be described the clinical scenario or physically enter the operating room to see the monitors and assess the situation, which tethers a physician to a workstation, limiting their usefulness in the perioperative environment. The present invention allows for a provider to roam the surgical center freely, and still have immediate, hands free access to every patient's physiologic monitors. Possibly most importantly, through the setting of appropriate monitor alarm parameters, allows for a physician outside of the operating room to be alerted of severe physiologic derangements by pushing notifications to their HMD. This allows for that physician's attention to be pulled to more critically ill patients while not viewing those routine cases that do not require their immediate attention. By focusing physician attention where it is needed, this device effectively multiplies a physician's presence. In critical situations, when seconds matter, the ability of a second provider to rapidly ascertain a clinical picture by viewing a patient's physiologic parameters while communicating with the care team can be the difference between a good and poor outcome. One can envision similar situations and the usefulness of such a tool in an intensive care unit or trauma bay.

This system also allows for hands free documentation of an electronic medical record (EMR). Current systems require a practice of action followed by remote documentation in critical care settings like an intensive care unit, operating room, or trauma bay. The first priority of the provider in these situations in stabilization of the patient through procedural and medical interventions with documentation taking a low priority in the moment. Oftentimes the medical record estimates a close approximation of the truth in these scenarios as the sheer frenzy of activity and provider focus on the patient distract from recollection of exact details. While providers attempt to recall the exact sequence of events and dosages of medication given in the moment, unless a person is designated specifically to recording events that are called out, reports generated in this fashion are inevitably flawed. Through integration with a hospital's EMR, the present system allows for real-time documentation of any medications given, procedures performed, or any narrative statement that the provider wearing a device wishes to record. No current system allows for this degree of accuracy for recording events in the acute care setting.

The veracity of a medical record can significant improve future quality of critical care by providing more detailed and accurate recounts for case review and development of future best practices.

### BRIEF DESCRIPTION OF THE DRAWINGS

The inventions of this application are better understood in conjunction with the following Figures and Detailed Description of the Invention. The various hardware and software elements used to carry out the inventions are illustrated in the attached drawings in the form of block diagrams and flow charts. For simplicity and brevity, the Figures and Detailed Description do not address in detail features that are well known in the prior art. However, to assure an adequate disclosure, the specification hereby expressly incorporates by reference each and every patent and other publication referenced in the application.

In all figures, a solid line denotes a wired connection, while a dashed line denote a wireless connection.

FIG. 1 shows a block diagram of the inventive wireless head mounted display and communication system.

FIG. 2 shows different functions supported by the companion application (computer program) of the inventive wireless head mounted display and communication system.

FIG. 3 is a block diagram of the inventive a head mounted display and communication unit (HMD).

FIG. 4 shows a perspective view of an embodiment of the head mounted device.

### DETAILED DESCRIPTION OF THE INVENTION

Definition:

An interface means a device or program enabling a component to communicate with a computer or another component.

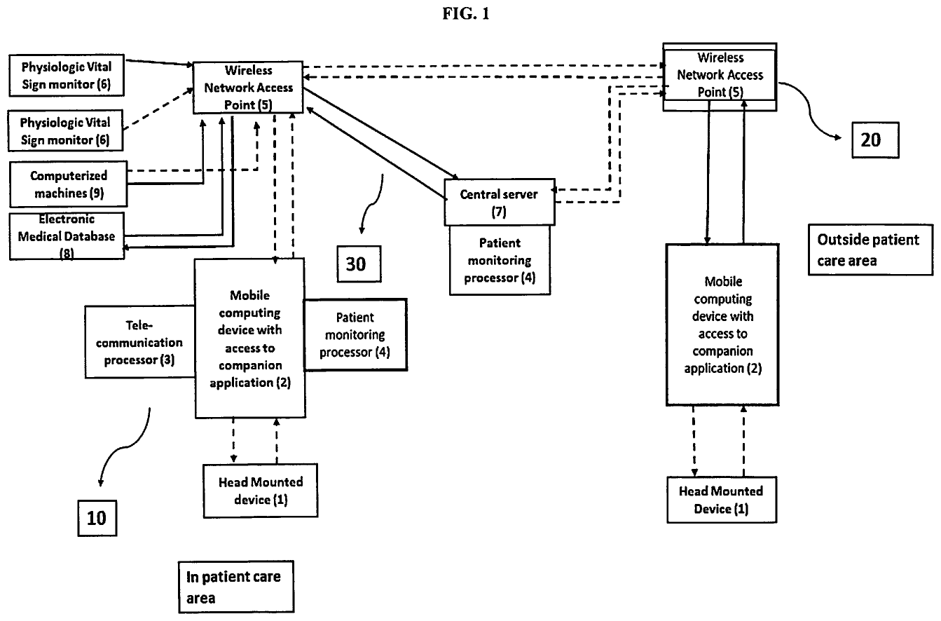

Turning now to FIG. 1, a head mounted display and communication system of the present invention comprises four primary components: a central server 7, a plurality of head mounted display and communication units 10, a patient monitoring assembly 6, or other computerized machines 9, and one or more wired 30 and/or wireless networks 20. While the head mounted display and communication units 10 are wirelessly connected to the head mounted display and communication system, the other components of the system may be communicatively connected by a wired or wireless network. To support data transfers between different components within a network, the central server, the patient monitoring assembly 6 or other computerized machines 9 may further comprise a communication port or an interface.

The patient monitor assembly comprises of a plurality of patient vital sign monitors 6. Each patient vital sign monitor 6 is capable of sensing a selected set of patient vital signs, include but not limited to, heart rate, blood pressure, blood oxygenation, patient temperature, blood pressure, pulse rate, and real-time electrocardiogram, and is capable of generating a patient parameters signal representing the values of the selected patient vital signs. Examples of the patient vital sign monitor include but not limited to an EEG, ECG, Pulse-oximeter, CO-oximeter, non-invasive blood pressure cuff, expired gas analyzer, and temperature probe. Patient vital sign monitors capture and record a patient's vital signs in real time, convert them into a patient parameter signal, and transmit the patient parameter signal to the central server 7 for storage or to selected head mounted display and communication unit 10 for display.

The patient monitoring and communication system may also comprises of one or more other computerized machines 9, which is communicatively connected to the central server 7 via wired or wireless network. These computerized machines 9 may include some of the machines routinely used in an operation room or an intensive care unit for the care of a patient whose primary function is not patient vital sign monitoring. Examples of a computerized machine 9 include but not limited to an anesthesia machine, a ventilator, a gas delivery assembly, an ultrasound or a fluoroscopy. The computerized machines often have sensors that are capable of sensing a selected set of parameters that is indicative of the machine's function or the status of a surgical procedure. Each computerized machine generates a machine signal representing values of these selected machine function parameters, and transmit said machine signal to the central server 7 by interfacing with the processors of the central server 7. In one embodiment, the computerized machine is a gas delivery assembly. The gas delivery assembly comprises one or more sensors that capable of sensing gas delivery parameters, such as flow rates, supply pressures, respiratory volumes, oxygen and carbon dioxide concentrations, which are all parameters indicative of the flow of a selected gas that is delivered to a patient. The gas delivery assembly is capable of generating a gas parameter signal representing real-time values of these gas delivery parameters, and transferring said gas parameter signal to the central server for storage and/or for transmission to one or more selected head mounted display and communication unit 10.

A central server or central data server 7 is equipped with at least one digital patient monitor processor 4, which is capable of receiving patient parameter signals from the patient monitor assembly 6 and/or machine signals from the other computerized machines 9 via a wired 30 or a wireless network 20. The central server 7 then converts these signals into alphanumeric or graphic data, and generating a continuous, or discrete data stream that may be transmitted and displayed on one or more selected head mounted display and communication unit 10. The central server 7 may further comprise an executable computer program that can grant access to one or more electronic medical database 8, that is stored on the central server 7 or on a separate data server or computer terminal, which is communicative connected to the central server via a wired or wireless network. The executable computer program, may further supports the retrieval, editing, transmission and storage of the medical records, and/or clinical data of a selected patient. A patient's real-time clinical data, including patient parameters and machine function parameters are collected by any aforementioned patient vital sign monitors within the patient monitoring assembly 6 and/or other computerized machines 9, converted into signals, and transmitted to the central sever 7, where the signals are processed into alphanumeric or graphic data, and selectively transmitted to desired head mounted patient display and communication units 10 for display. The central server 7, upon requests from the users via their head mounted patient display and communication units, may also selectively retrieve a patient's medical record, and transmit it to a selected head mounted display and communication unit 10, where the medical record is displayed in the field of vision of the care provider wearing that head mounted display and communication unit. The interface between the central data server 7 and each of the plurality of head mounted display and communication unit 10 is supported by a wireless network, allowing the user (care provider) wearing the head mounted display and communication unit to freely move throughout the facility supported by the network while still have access to patient medical record and clinical data such as patient medical history, and real-time patient vital signs, machine function parameters, medications, and physician instructions.

The head mounted display and communication unit 10 of the inventive system interfaces with the central server 7 via a wireless network 20. Through this interface, the head mounted display and communication units 10 can transmit (sent and receive) audio and visual data steams to the central sever 7, and any other computers, or computerized devices that are connected the central server 7. The care provider wearing the head mounted display and communication unit can remotely retrieve clinical data of a selected patient or a patient's medical record without the use of another computer or move around.

A head mounted display and communication unit 10 of this invention comprises a mobile computing device 2, and a head mounted device 1. The mobile computing device 2 may be a cell phone or a small computerized device, which comprises a combined patient monitoring and telecommunication processor or a pair of stand-alone patient monitoring processor 3, and a telecommunication processor 4. While the telecommunication processor supports visual and audio communication between different the head mounted display communication units 10, the patient monitoring processor 3 allows the mobile computing device to directly retrieve and process patient parameter signals or machine signals from selected patient vital sign monitors 6 or other computerized machines 9 that are connected to the same wireless network without the involvement of the central server 7. The patient monitoring processor also allows the mobile computer device to transmit (sent and receive) visual and audio data between head mounted device 1 and the central server 7.

The mobile computing device 2 can selectively interfaces with the central server 7, patient vial sign monitors 6 or other computerized machines 9, and other head mounted display and communication units 10 within the same network 20 using a companion application or companion computer program. This companion application or computer program may be a mobile application downloadable to the mobile computing device 2, or an executable computer program hosted on the central server 7 or a web server that is communicatively connected to the central server 7. The companion computer program enables data transfer and communication between different components of the system.

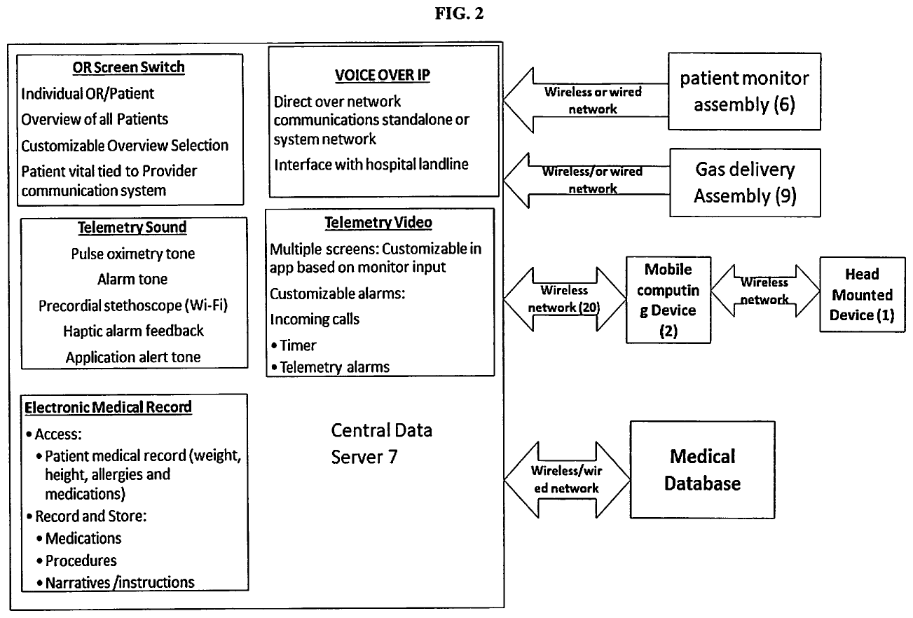

FIG. 2 shows an embodiment of the present invention, and different commands may be executed by the companion computer program or companion application. In this embodiment, the companion application is an executable computer program stored on the central server 7, and the medical database is stored on a separate server, which interfaces with the central server 7 via a wired or wireless network. Patient monitoring/gas delivery signals of one or more patient are sent from the patient monitoring assembly 6 and gas delivery assembly to the central server 7 via a wireless or wired network. A user use the wireless head mounted display and communication unit 10 and its companion application to execute different commands.

The companion computer application enables a user to access electronic medical record of a particular patient, and display them on the head mounted device selected from the plurality of head mounted display and communication units. The user can retrieve, edit and save that patient's medical records via wireless connection to the central server via verbal or touching command. For example, a care provider can use his head mounted display and communication unit 10 to issue voice activated commands to the central data server 7, which execute the companion program, and have medical records of one or more patients displayed on the care provider's head mounted device 1. The care provider can also edit a patient record using key pad of the mobile computing device. The companion computer program also allows users to selectively display monitoring data of one or more patients on the head mounted display and communication units 10. For example, a care provider can select to display real-time patient monitoring and gas delivery data streams associated one or more patients on his head mounted device. Using the companion application, a care provider can also customize the type and format of patient monitoring and gas delivery information to be displayed (multi-monitors vs. single machine; complete patient monitoring parameters vs. selected patient monitoring parameters). The care provider can uses their head mounted display and communication unit 1 to active the companion program on the central server and selectively communicate with other care providers. For example, a user may call up a screen to be displayed on his head mounted device 1, such as a dial up screen or telemetry video/audio screen. Using the dial up screen, the user can selectively communicate with other care providers via their head mounted display and communication unit 10. The companion program may also support direct audio/video (A/V) communications via a wireless network or within the existing hospital communication system such as a landline or a standalone device. For example, a user can use his head mounted device to capture screen display of a patient monitor or video of a patient, and transmit these A/V signals to another user's head mounted display and communication unit. The companion program may further enable the generation and selection of different audio/visual/tactical signals, which include but not limited to pulse oximetry tone, alarm tone, sound of precordial stethoscope (Wi-Fi), Haptic alarm feedback sound or application alert tone. For example, a care provider, via the companion program, can set up an audio alarm when a selected patient clinical parameter exceed the threshold or a tactic alert for incoming call.

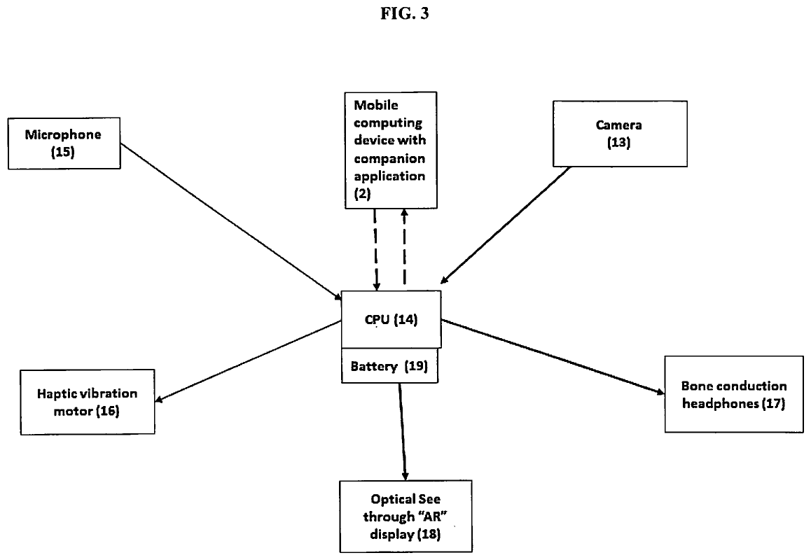

The end user experience of the inventive head mounted display and communication system is primarily enabled by the interaction through a user's mobile computing device (2) and their head mounted device (1). The head mounted device 1 comprises of a combination of the components shown in FIG. 3, which include: a microphone 15, a haptic tactical sensor, such as a haptic vibration motor 16, an optical display 18, a video camera 13, a headphone 17, a digital processer 14 and a battery 19. The head mounted device 1 is powered by a battery 19 and controlled by a computer processor 14, which is capable of transmitting (receives and sends) audio/visual (AN) signals to and from the mobile computing device 2 via a wireless/wired network. The microphone 15 allows the users to issue verbal (voice) commands to the head mounted display and communication system. Using verbal commands, a user can customize the information to be displayed on his or her optical display 18, set up different audio, visual or tactical alarms, and enable hands free communications with different users within the network. Verbal commands is processed and executed by the companion application (or companion computer program). The requested visual/audio/tactical signals are then relayed back to the head mounted device via mobile computing device.

For example, a supervising physician can issue verbal commands via his head mounted display and communication unit 10 to request vital signs or medical record of one or more patients under his care to be displayed on his head mounted device. A physician extender can also verbally dial a supervising physician for consultation.

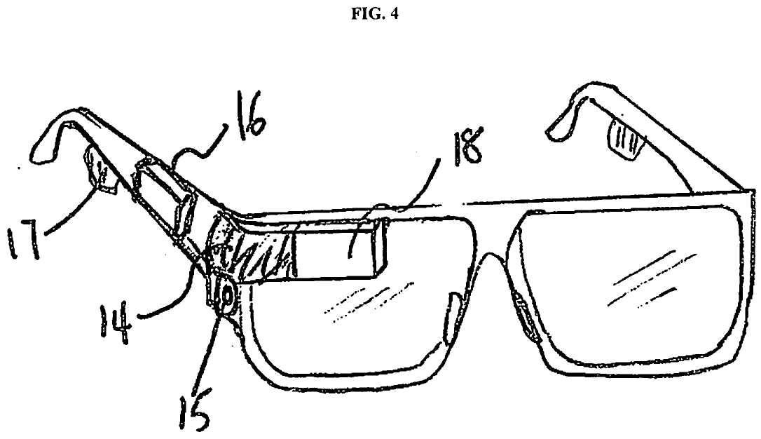

FIG. 4 shows a perspective view of an embodiment of the head mounted device 1. In this embodiment, the optical display is an optical see through display 18 and the headphone is a bone conduction headphone 17, both components are chosen to provide the user continuous situational awareness of his or her clinical environment while augmenting the user's audio and visual reality with overlaid data. A see through “AR” display 18 provide the user with selected views of a patient's clinical data (vital signs and machine parameters), and medical records while allow the user to maintain constant visual of things occurring in his direct vision. This function enables a care provider to view selected patient clinical data without diverting his/her sight from the procedure that he or she is performing. A bone conduction headphone 17 can provide clear and continuous audio signals to a user in noisy environment. For example, a care provider may select to receive audio signals such as pulse oximetry tones and alarm sounds, while working with an emergency team. The head mounted device 1 may interfaces with the mobile computing device 2 through wireless connection using standard wireless communication protocols such as Bluetooth. A video camera 13 can be used to capture the screen display of a particular patient vital sign monitor 6 or the video of the patient. A physician can thus make a direct real-time assessment of a patient remotely based on the images or videos captured by another user's head mounted device 1, and provide time-sensitive instructions to other care providers in critical situations.

In one embodiment, a head mounted device (HMD) is a glass like device wore by members of a care team, which has access to a web based companion computer program. This web based companion computer program can be hosted either from the central data server 7 or individual computer terminals within a secure wireless network 20. Alternatively, the companion computer program is machine based and accessible to every mobile computing device on the wireless network, which relays audio and visual (A/V) signals to/from the head mounted device 1. It also processes voice commands from the head mounted device 1, and modulates its A/V display on the head mounted device 1 accordingly. The mobile device can also serve as a voice-over-IP (VoIP) communications hub 5 for the inventive system. A voice-over-IP (VoIP) network allows individual users to communicate through their head mounted display and communication unit 10, enabling both voice control of their HMD 1 and VoIP connectivity 10 with other providers on the network.

### EXAMPLE 1

### A Prototype Head Mounted Display and Communication System

A prototype head mounted display and communication system is an anesthesiology monitoring and communication system, comprising:

(a) a patient monitor assembly comprises a plurality of patient vital sign monitors, said patient vital sign monitor senses patient vital parameters of a patient associated with said patient vital sign monitor, and generates a patient parameter signal, which represents a value of said patient vital parameters;

(b) a gas delivery assembly comprises a plurality of gas delivery sensors, said gas delivery sensor senses gas parameters indicative of flow of a selected gas delivered to a patient, and generating a gas parameter signal representing a value of said gas parameters;

(c) a central data server comprising: (i) a digital patient monitor processor having said plurality of gas delivery sensors and said plurality of patient vital sign monitors interfaced thereto, said digital patient monitor processor executing a program to selectively process said patient parameter signal and said gas parameter signal and generating alphanumeric and graphic data representing the patient vital parameters and gas parameters from one or more said plurality of patient vital sign monitors or gas sensors; and (ii) a patient record management database, said patient record management database executing a program to selectively retrieve, edit, store and transmit a patient's record;

(d) a head mounted device, said head mounted device comprising: (i) a microphone; (ii) a haptic tactic alarm; (iii) a bone conducting headphone; (iv) a digital camera; (v) a display; and (vi) a battery;

(e) a mobile computing device comprising

(i) a display controller interfaced to said central data server and said head mounted device and communication device, and generating a display signal to presented on said head mounted device representing
(aa) alphanumeric and graphic data generated by said digital patient monitor processor including indicia representing said gas parameter and said patient vital parameters; and
(bb) said patient record retrieved by said patient record management database; and
(ii) a communication controller interfaced with said central data server and said head mounted display and communication devices and supporting video and audio communications between a plurality of said head mounted device.

A user may use voice or touch command to selectively retrieve display a patient's record or vitals and gas delivery parameters on his head mounted device or selectively communicate with other users within the network.

What is claimed is:

1) A head mounted display and communication system, comprises:
a) a central server;
b) a plurality of head mounted display and communication units, interfaced with the central server;
c) a patient monitor assembly, or other computerized machine communicatively connected to the central server, wherein
(i) said patient monitoring assembly is capable of sensing vital signs of one or more patients and generating a patient parameters signal for each said patient representing the values of said patient's vital signs; and
(ii) said other computerized machine is capable of sensing a selected set of parameters said the machine, that is indicative of the machine's function and generating a machine signal; and
d) a wireless network;
wherein said central server execute a computer program to selectively display patient parameter signals of one or more patient, and/or machine signals on one of the plurality of head mounted display and communication units.

2) The system of claim 1, said patient monitor assembly comprises of a plurality of patient vital sign monitors, said patient vital sign monitor is capable of sensing a selected set of patient vital signs of an associated patient.

3) The system of claim 2, wherein said patient vital signs include heart rate, blood pressure, blood oxygenation, patient temperature, blood pressure, pulse rate, or real-time electrocardiogram.

4) The system of claim 2, where said patient vital sign monitors are selected from the group consisting an EEG, ECG, Pulse-oximeter, CO-oximeter, non-invasive blood pressure cuff, expired gas analyzer, and temperature probe.

5) The system of claim 1, wherein said computerized machines include an anesthesia machine, a ventilator, a gas delivery assembly, an ultrasound or a fluoroscopy.

6) The system of claim 1, wherein said computerized machine is a gas delivery machine and said machine parameter include flow rates, supply pressures, respiratory volumes, oxygen and/or carbon dioxide concentrations.

7) The system of claim 1, wherein said system further comprises a medical database.

8) The system of claim 7, wherein said central server can execute a computer program to selectively retrieve a patient's medical record from said medical database, transmit and display said medical record on one of said plurality of head display and commutation unit.

9) The system of claim 1, wherein said central server comprise at least one digital patient monitor processor, said digital patient monitor processor is capable of
i) receiving patient parameter signals from the patient monitor assembly and/or machine signals from the other computerized machines via a wired or a wireless network;
ii) converting said patient parameter signals or machine signals into alphanumeric or graphic data, and
iii) generating a continuous, or discrete data stream that is transmitted and displayed on one or more of the plurality of head mounted display and communication units.

10) The system of claim 1, wherein said head mounted display and communication unit comprises a mobile computing device, and a head mounted device.

11) The system of claim 10, wherein said mobile computer device further comprises a combined patient monitoring and telecommunication processor or a pair of stand-alone patient monitoring processor, and a telecommunication processor, wherein
i) said telecommunication processor enables visual and audio communication between said head mounted display communication units; and
ii) the patient monitoring processor enables the retrieval and process of patient parameter signals or machine signals from selected patient vital sign monitors or other computerized machines that are connected to the wireless network.

12) the system of claim 1, wherein said head mounted device further comprises:
i) a microphone;
ii) a haptic tactical sensor;
iii) an optical display;
iv) a video camera;
v) a headphone;
vi) a digital processer; and
vii) a battery.

13) the system of claim 1, wherein said system further comprises a companion application, said companion application contain executable computer programs that enables selective display continuous, or discrete data stream of one or more patients or communications between selected head display and communication unit.

14) An anesthesiology monitoring and communication system, comprising:
(a) a patient monitor assembly comprises a plurality of patient vital sign monitors, said patient vital sign monitor senses patient vital parameters of a patient associated with said patient vital sign monitor, and generates a patient parameter signal, which represents a value of said patient vital parameters;
(b) a gas delivery assembly comprises a plurality of gas delivery sensors, said gas delivery sensor senses gas parameters indicative of flow of a selected gas delivered to a patient, and generating a gas parameter signal representing a value of said gas parameters;
(c) a central data server comprising
(i) a digital patient monitor processor having said plurality of gas delivery sensors and said plurality of patient vital sign monitors interfaced thereto, said digital patient monitor processor executing a program to selectively process said patient parameter signal and said gas parameter signal and generating alphanumeric and graphic data representing the patient vital parameters and gas parameters from one or more said plurality of patient vital sign monitors or gas sensors; and
(ii) a patient record management database, said patient record management database executing a program to selectively retrieve, edit, store and transmit a patient's record;
(d) a head mounted device, said head mounted device comprising:
(i) a microphone;
(ii) a haptic tactic alarm;
(iii) a bone conducting headphone;
(iv) a digital camera;
(v) a display; and
(vi) a battery;
(e) a mobile computing device comprising
(i) a display controller interfaced to said central data server and said head mounted device and communication device, and generating a display signal to presented on said head mounted device representing
(aa) alphanumeric and graphic data generated by said digital patient monitor processor including indicia representing said gas parameter and said patient vital parameters; and
(bb) said patient record retrieved by said patient record management database; and
(ii) a communication controller interfaced with said central data server and said head mounted display and communication devices and supporting video and audio communications between a plurality of said head mounted device.

2. The system of claim 1, wherein patient record management database and said digital patient monitor processor are controlled by said head mounted device.
